(19) United States
(12) Patent Application Publication
(10) Pub. No.: US 2005/0054938 A1
(43) Pub. Date: Mar. 10, 2005

(54) Method and apparatus including altimeter and accelerometers for determining work performed by an individual

(76) Inventors: Thomas C. Wehman, Cupertino, CA (US); Serjan Nikolic, San Francisco, CA (US); Milos Backovic, Los Altos, CA (US); Peter Muller, Woodside, CA (US); Jim Lovewell, San Leandro, CA (US); Katie Peuvrelle, San Jose, CA (US)

Correspondence Address: FENWICK & WEST LLP, SILICON VALLEY CENTER, 801 CALIFORNIA STREET, MOUNTAIN VIEW, CA, 94041, US

(21) Appl. No.: 10/901,675

(22) Filed: Jul. 28, 2004

Related U.S. Application Data
(60) Provisional application No. 60/491,162, filed on Jul. 29, 2003.

(51) Int. Cl. A61B 5/02

(52) U.S. Cl. 600/483; 600/595

(57) ABSTRACT

Method and calculations determine an individual's, or several individuals' simultaneous rates of oxygen consumption, maximum rates of oxygen consumption, heart rates, calorie expenditures, and METS (multiples of metabolic resting rate) in order to determine the amounts of work that is performed by the individual's body. A heart monitor measures the heart rate, and an accelerometer measures the acceleration of the body along one or more axes. An altimeter measures change in altitude, a glucose monitor measures glucose in tissue and blood, and thermometers, thermistors, or thermocouples measure body temperature. Data including body fat and blood pressure measurements are stored locally and transferred to a processor for calculation of the rate of physiological energy expenditure. Certain cardiovascular parameters are mathematically determined. Comparison of each axis response to the individual's moment can be used to identify the type of activity performed and the information may be used to accurately calculate total energy expenditure for each physical activity. Energy expenditure may be calculated by assigning a separate proportionality coefficient to each axis and tabulating the resulting filtered dynamic acceleration over time, or by comparison with previously predetermined expenditures for each activity type. A comparison of total energy expenditure from the current activity is compared with expenditure from a previous activity, or with a baseline expenditure rate to assess the level of current expenditure. A measure of the individual's cardio-vascular health may be obtained by monitoring the heart's responses to various types of activity and to total energy expended.

### RELATED APPLICATION

This application claims the priority benefit of pending provisional application Ser. No. 60/491,162, filed on Jul. 29, 2003 by T. Wehman et al., which is incorporated herein in the entirety by this reference thereto.

### FIELD OF THE INVENTION

The present invention pertains to the field of physiological monitoring and more particularly, to a method, apparatus and calculations for determining an individual's or several individuals' rates of oxygen consumption, maximum rates of oxygen consumption, heart rate, and calorie expenditure. Glucose level, percent body fat, blood pressure, body and external temperature may all be analyzed in order to measure the amount of work performed by the individual or several individuals. The device may be used to simultaneously monitor separate individuals, for example, racers, or a mother and her fetus' heart rate during activity that she performs.

### BACKGROUND OF THE INVENTION

Increase in physical activity and total body energy expenditure (EE) are directly related to the improvement in outcomes in chronic diseases, better weight management and prevention of obesity, and overall increate of longevity. Current methods of EE assessment have shortcomings that may significantly impact the success of therapeutic treatments, programs for body weight control and achievement of cardiovascular or general physical fitness. In addition, most EE measures and indexes and not feasible to use in free-living situations, or outside laboratories or specialized fitness facilities. This unmet need for accurate assessment of physical activity and body EE is explicitly stated in a Surgeon General's report on Physical Activity and Health.

Conventional methods of estimating total body EE use questionnaires about participation in sports and programmed exercise. However, they are inherently subjective and imprecise, and frequently there is a major uncertainty over what exactly is being measured. Although the questionnaires correlate modestly with other assessments of vigorous physical activity, the measurement of moderate or light activity is less accurate.

Objective measures of EE are determined by using various instruments and devices. The gold standard for measuring body EE in free-living people is double-labeled water method which involves administration of hydrogen and oxygen isotopes and determination of washout kinetics of both isotopes. An alternative “gold-standard” method is a portable gas analyzer which EE can precisely monitor all expired and consumed gases from a subject in a free-living environment.

It is commonly understood that there is a direct physiological relationship between EE and heart rate (HR) and many attempts have been made to use HR measurement for estimating physical activity, oxygen consumption and body EE. HR measurement is broadly used because it is practical and relatively easy to monitor. However, determination of EE from HR measurement is not reliable or accurate because the relationship between HR and EE is dependent upon physical fitness level and weight of the individual. For example, HR measurements grossly overestimate EE in deconditioned and overweight individuals and have a tendency to underestimate EE with increases in fitness. HR is altered by emotional stress and anxiety which may introduce significant error (>50%) in estimating EE.

The HR response may lag at the beginning or end of activity, thus EE determination in an intermittent activity or exercise may promote large measurement errors. In addition, disease status has an important influence on the ability of HR to predict EE, such as in patients with chronic heart failure (CHF). Patients suffering from heart failure have “blunted” HR responses to exercise that make it difficult to estimate true EE based upon HR measurements. This presents a significant problem since exercise training is an integral part of heart failure treatment and is recommended by both American Heart Association and American College of Cardiology.

A number of motion sensors have been used to objectively estimate body movement and body EE. Use of various pedometers and accelerometers is practical and inexpensive, and may provide viable alternative to the EE estimation based on HR. Pedometers for example, are generally not accurate, since they are not able to detect different activities and generally underestimate EE.

Accelerometry promotes more accurate measurement of EE in able-body individuals, without the need for individual calibration. The improvement of the body EE determination in subjects performing a walking test is achieved by adjusting for body weight and by measurement of the slope of the walking surface. Accurate application of accelerometers to measuring EE is also related to the type of the sensor and sensor location on the individual.

There is general tendency of accelerometry to underestimate EE in free-living conditions, mostly due to the lack of upper body movement measurements and possible load carriage. Estimations of the body EE are dependent upon the accuracy of accelerometers to represent various activities and exercise which require unique algorithms for each type of physical activity.

HR and motion sensors each provide a physical activity assessment and an estimate of the body EE. Because each has inherent limitations, the simultaneous use of HR and motion sensors may increase the accuracy of EE estimates. Preliminary studies have demonstrated that the prediction of EE based on combination of the HR and activity measurements is superior to EE estimates based on any individual component.

Therefore, the reliable measurement of total daily energy expenditure in free-living individuals requires a practical, relatively inexpensive method that will at least combine HR and activity measurements, and account for age, weight, fitness level and conditions of activity (i.e. slope of the walking surface or load carriage).

### BRIEF DESCRIPTION OF THE DRAWINGS

FIG. 1 is a graph illustrating exemplary heart rates for individuals having various degrees of physical conditioning, during a physical activity;

FIG. 2 is a block schematic diagram of monitoring apparatus according to one embodiment of the present invention;

FIG. 3 is a block schematic diagram of a system for monitoring one or more individuals with monitoring apparatus according to the illustrated embodiment of FIG. 2;

FIG. 4 is a flow chart illustrating operation of the invention in accordance with one embodiment; and

FIG. 5 is a flow chart illustrating operation of the invention in accordance with another embodiment.

### SUMMARY AND DISCLOSURE OF THE INVENTION

In accordance with the present invention, methods and calculations determine an individual's or several individuals' simultaneous rates of oxygen consumption, maximum rates of oxygen consumption, heart rates, calorie expenditure and multiples of metabolic resting rate (METS) in order to measure the amounts of work performed by the individual or several individuals “simultaneously”, as during training or racing activities. A heart monitor is used to measure the heart rate, an accelerator aligned along each of multiple axes measures accelerations. An altimeter is used to correct the accelerometer by determining if the user is proceeding uphill or downhill. The heart rate and acceleration outputs are stored in a local storage device, are treated mathematically, and are displayed in real time, and can be downloaded to a local base station. After the base station receives the outputs, the heart monitor and accelerometers are available to take more measurements or perform more mathematical or graphical outputs. The base station, meanwhile, is available to upload the outputs to a central processor as a clearinghouse for processing. More specifically, the acceleration outputs are collected and mathematical algorithms are employed to initially convert the outputs into motion information and then into activity information. The heart rate and activity information are then graphed on the same or similar time base for determining their relationship to calculate cardiovascular response to the activity. Comparison to previous activity sessions, or base line energy expenditure, or to tabulated “normal, health” responses from certain populations can be made instantaneously.

Results of energy calculations for any of the monitored individuals may be transmitted simultaneously via a multi-user transceiver such as “Blue Tooth” or similar technology. Comments and resultant data-derived parameters may be displayed audibly and may be initiated by voice commands inputs using pre-programmed words or phrases with above-mentioned multi-user technology.

Furthermore, voice commands and the verbal responses may be incorporated into an audio or visual recorder which may be simultaneously playing music or giving rhythmic beat or other audible information to the individual.

Heart rate may be obtained via electrical impulses or audiology. Exercise goals may be determined from previous sessions, or pre-selected, or may be compared in real time during a competitive physical activity among multiple individuals. An audible or vibratory indication may be given when these goals are met or exceeded.

All data and results may be retrieved by audible signals such as specific words or phrases for each individual being monitored. All data and results may also be displayed or reported audibly by better commands for each individual monitored.

In accordance with the present invention, methods and calculations for determining an individual's rate of oxygen consumption, maximum rate of oxygen consumption, heart rate and calorie expenditure are set forth in Appendix I hereof in order to measure the amount of work performed by the individual's body. The methods and calculations of the invention allow for the heart rate and acceleration measurements to be taken at the location where the activity would normally take place, such as in a gymnasium or a swimming pool, on a track, a court or a field, or at home. Furthermore, the methods and calculations in accordance with the present invention allow for the activity to take place under normal conditions such as running, hiking, bicycling, kayaking and the like.

The data and calculations can be made to be sports or exercise specific. Data and resultant energy expenditure calculation can be done in a “free-living environment” since all calculated data can be obtained under those directions. This information can be helpful for management of weight loss and blood glucose levels in diabetics or pre-diabetics.

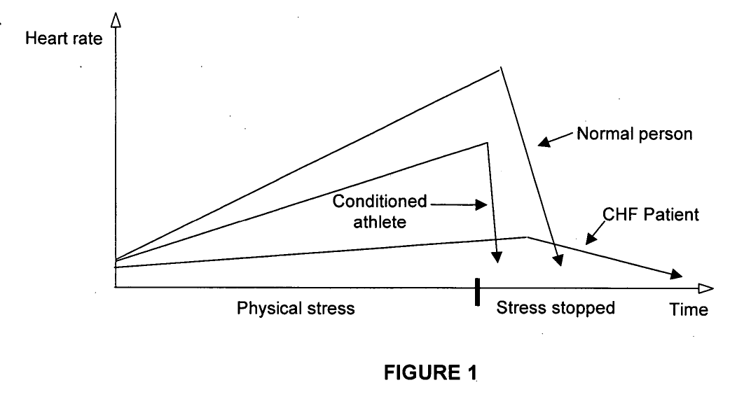

Measures of recovery rate utilizing heart rate data is useful for gauging the cardiovascular health of an individual, as illustrated in FIG. 1. As shown, the heart rate of a normal person may increase with time during a physical stress or activity, and then decrease over time following cessation of the stress or activity. The heart rate of a conditioned athlete may increase less with time during an activity and decrease more rapidly following cessation of the activity, in contrast to the heart rate of a normal person. In contrast, a chronic heart failure (CHF) patient commonly displays a heart rate that increases very slowly with time during a physical activity, and that decreases very slowly following cessation of activity, as shown.

The following quotient is also helpful for determining cardiovascular health:

$$Health Quotient = HQ = [ \frac{Energy Expenditure {( ergs or calories )}_{T}}{Total heart Beats} ] ;$$

where T=Time period for Energy Expenditure over some range of activity

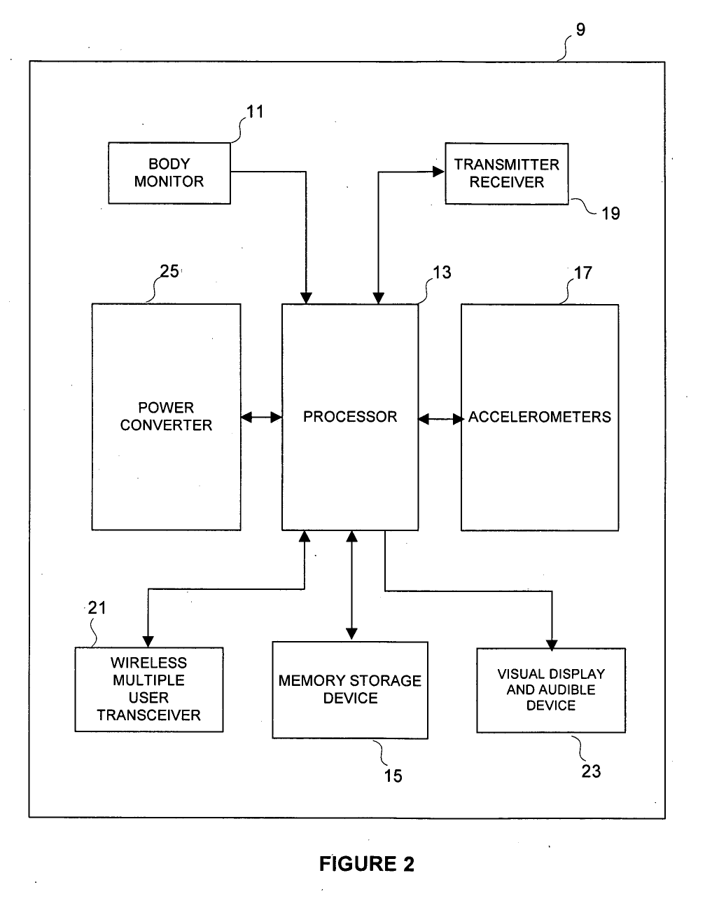

Referring now to. FIG. 2, there is shown a block diagram of a Total Energy Expenditure Monitor (TEEM) in accordance with one embodiment of the present invention. The monitor 9 includes one or more body sensors 11 such as for sensing heart rate, temperature, and the like, that supplies sensor data to processor 13 for storage 15 and processing in a manner as set forth in Appendix I hereof. In addition, processor 13 receives acceleration data from multiple accelerometers 17 oriented about an individual's body substantially along orthogonal axes. Also, the processor 13 receives and transfers data and calculations via transmitter/receiver 19 and/or multiple-user transceiver 21 for data communications with a base station or other similar monitors on individuals engaged in a similar activity, such as foot-racing for performing comparative analyses on similar data from individuals similarly engaged in a physical activity together. The processor also produces visual and/or audible outputs 23 available to the individual user as pacing information or reports of calculated data such a heart rate, body temperature, lapsed time of activity, or the like. Also, the accelerometers 17 may include one altitude-sensor or altimeter for detecting change of elevation with time of movement, for example, uphill or downhill, to provide slope information. And, power converter 25 may include battery primary power source, with back-up charger circuitry, or the like, for powering the processor and all attached peripheral devices.

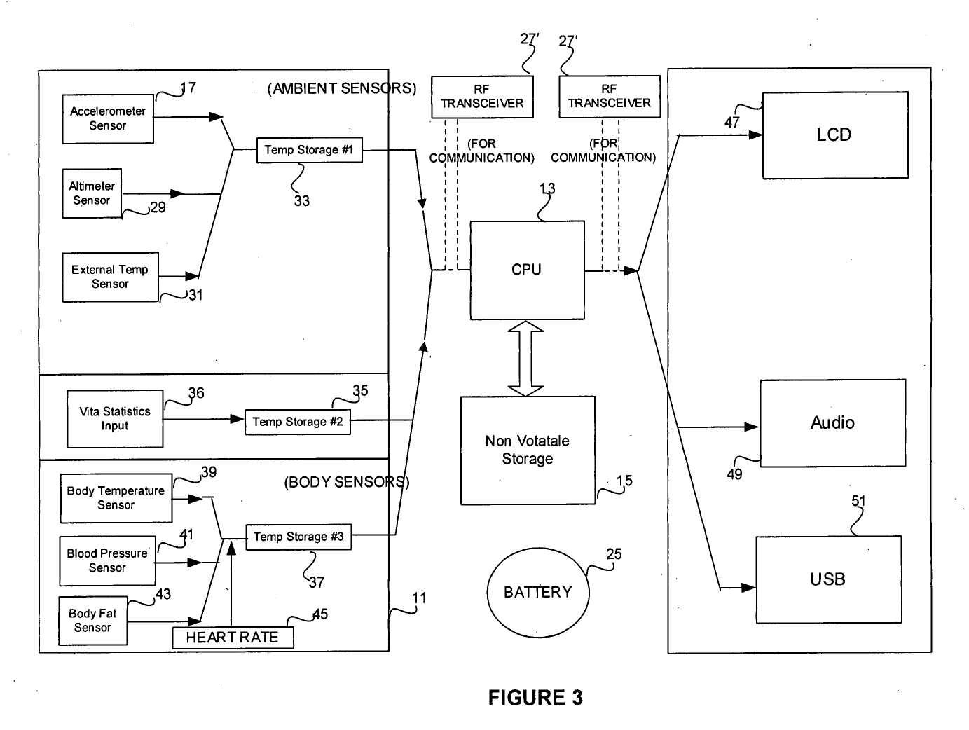

Referring now to the block schematic diagram of FIG. 3, the processor 13 of the TEEM 9 is shown connected (either directly or via wireless communication channel 27) to receive data from accelerometer sensors 17 and from an altimeter sensor 29 and from an ambient temperature sensor 31. The data may be temporarily stored 33 for supply to the processor 13, as required. In addition, there is a temporary storage module 35 for storing 36 individual data such as age, weight, sex, body mass index, and the like, as may be entered via keyboard or external communication, or the like.

Also, the processor 13 is connected to receive (either directly or via wireless communication channel 27) data from the temporary storage 37 as required regarding the sensed 39 body temperature, the sensed 41 blood pressure, and sensed 43 body fat (such as via electrical conductance measurement), and the sensed 45 heart rate.

In addition, the processor 13 is connected either directly or via a wireless channel 27 to a visual display 47, and to an audio display or annunciator 49, and to an universal serial bus (USB) 51 for data transfers between the processor 13 and a base station or other computer. Non-volatile memory 15 stores operating algorithms, for example, to process supplied data in accordance with the procedures and calculation set forth in Appendix I. And, battery 25 may conveniently power the TEEM during operation as a portable module.

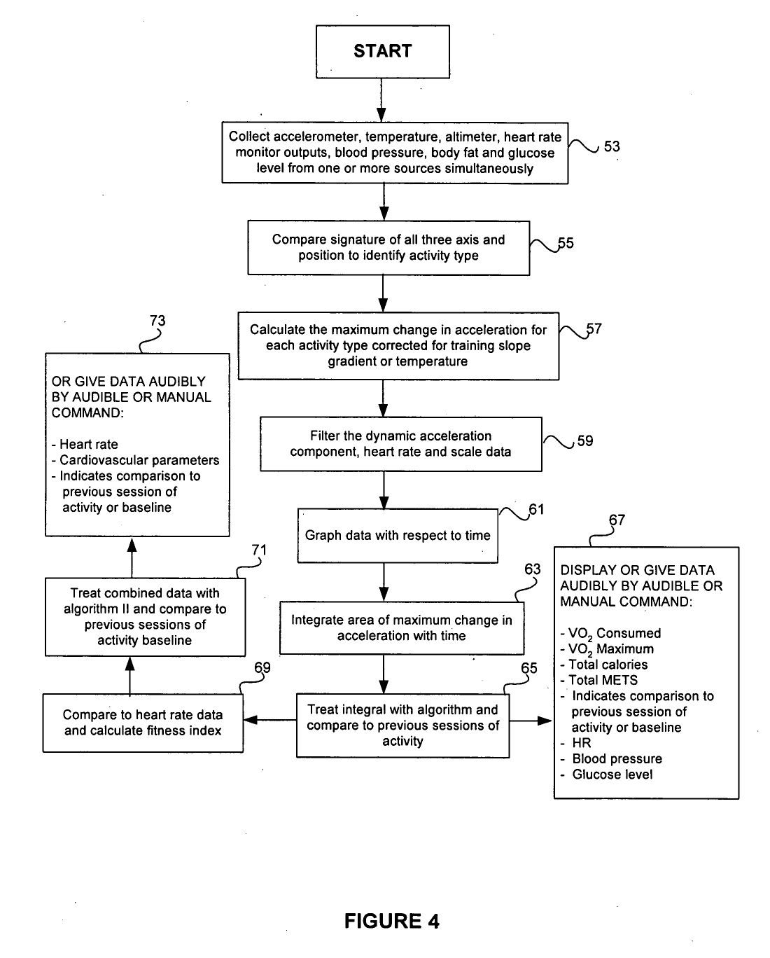

In operation, the system of FIG. 3 operates in one mode in accordance with the present invention as illustrated in the flow chart of FIG. 4 using pre-filtering of dynamic acceleration data. At the start, the sensors supply and the memory modules store 53 data pertaining to accelerations, temperature, altitude, heart rate, blood pressure, glucose level and the like, from sensors disposed about an individual's body in a manner as previously described herein. The particular ‘signature’ of acceleration and altimeter data during an individual's activity is indicative of an activity (e.g. rowing vs. jogging). These data are gathered to identify 55 the particular activity, and the maximum change in acceleration is calculated 57 for each activity type and is corrected for slope or ambient temperature.

The acceleration data is filtered 59 (e.g. via peak detection or average per incremental time sample, or the like), and normalized or scaled for age, temperature, altitude, or the like, and the resultant data may be graphed 61, or accumulated in storage, in order to integrate 63 the maximum change of acceleration with time. Various comparisons 65 of the integrals may be made against previous sessions of the same activity to determine improvement in the individual's performance to produce various outputs 67, as indicated. In addition, a “fitness index” may be calculated 69 and computed in accordance with the Appendix I for various comparisons 71 to provide displays or audible outputs 73, as indicated.

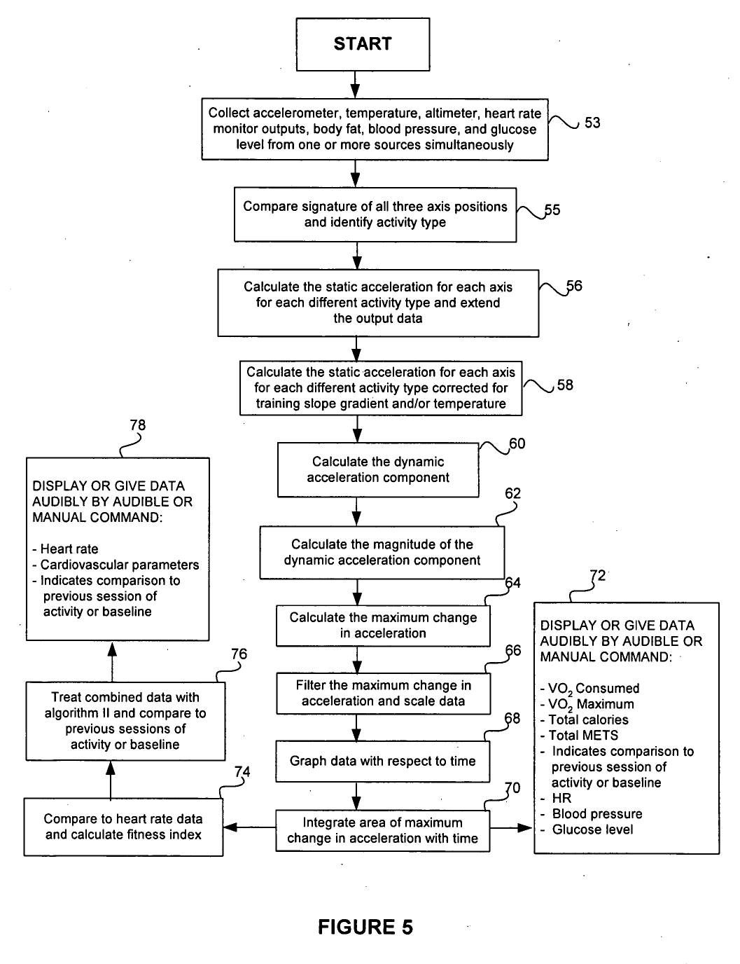

Referring now to the flow chart of FIG. 5, there is illustrated another operating mode of the present invention using post-filtering of dynamic acceleration data. At the start, the sensors supply and the memory modules store 53 data pertaining to acceleration, temperature, altitude, heart rate, blood pressure, glucose level and the like from sensors disposed about an individual's body in a manner as previously described herein. The “signature” of acceleration and altimeter data during an individual's activity indicates the activity 55. Then, the static acceleration data for each axis of acceleration is filtered (for the identified activity) and data for subsequent intervals (even beyond cessation of the activity) are extended or replicated 56, and the static acceleration data for each acceleration axis is corrected 58 for slope (e.g. rate or change of altitude), temperature, and the like. The dynamic acceleration (i.e., without the static component) is calculated 60, and the magnitude of the dynamic acceleration component is calculated 62. The maximum change in acceleration is calculated 64 and filtered 66 to yield data for graphing 58 or accumulating in storage, that is then integrated 70 over time to yield various outputs 72, as indicated.

In addition, a ‘fitness index’ may be calculated 74 and computed in accordance with the Appendix I for various comparisons 76 to provide displays or audible outputs 78 as indicated.

### APPENDIX I

Definitions:

1. TEEM=Total Energy Expenditure Measurement
2. Acceleration (A)=Distance/Time²=D/T²
3. Force (F)=Mass×Acceleration=M×A
4. Mechanical Work (Wₘ)=Force×Distance=F×D or by substituting (3) into this equation for F: Wₘ=M×A×D
5. Maximum Change in Dynamic Acceleration (MCDA) is a mathematical treatment of the TEEM data which doesn't change acceleration values or dimensional units.
6. Total Maximum Change in Dynamic Acceleration [(MCDA)_{T-Area}] is the sum of the area under each (MCDA) Time (T) curve and is equal to the integral, ∫yᵢdx, where yᵢ=height of a rectangle segment, (i), with infinitesimal base width, dx. After integration, [(MCDA)_{T-Area}] is equal to (Σyᵢ)(x); or since: (Σy₁) is proportional to (MCDA) and (x) proportional to (T), then by substitution: [(MCDA)_{T-Area}] is proportional to (MCDA)(T).
7. VO₂ Max is measured maximum oxygen consumption rate of an individual during an aerobic stress test and is usually expressed as VO₂/M.
   Assumptions:
8. MCDA has the same units and is proportional to acceleration (A).
9. Distance (D) on a treadmill is proportional to Time (T).
10. The product (MCDA)×(T) is proportional to the product (MCDA)×(D) since (D) is proportional to (T).
11. During a VO₂ test, oxygen consumption increases with time in a regular manner until VO₂Max and can be approximated mathematically as a triangle with the base (B) equal to (time) and the height (H) equal to (oxygen consumption rate). Then the O₂Max, equals the maximum height of the triangle.
12. During the VO₂ test, total oxygen consumption was calculated from the sum of the average consumption rate for each minute interval. The average oxygen consumption for each minute was calculated by adding the rate at the end of the previous minute to the rate at the end of the present minute and dividing by 2. At the start of the first minute, the standard ‘at rest rate’ of 3.5 ml/min/Kg was used. The amount of O₂ consumed for the last interval was calculated as its fractional proportion of a minute, still using the average rate for that interval.
   Resultant Equations:
   Total work:
13. From (4) above, Mechanical Work (W_{M}) from the TEEM data=[M×A×D].

Substituting the equivalences from (7) & (8) above, we obtain: W_{M} is proportional to [(M)×(MCDA)×(T)].
14. Total Mechanical Work (W_{M})_{T} for the duration of each test=[(M)×(MCDA)×(T)]_{T} from (10) above. BY substitution from (6) above, (W_{M})_{T} is then proportional to: [(M)×(MCDA)_{T-area}]
15. Biological Work (W_{B}) is proportional to (VO₂) consumed. Total Biological Work (W_{B})_{T} is proportional to Total (VO₂) consumed.
16. Equating (11) to (12) above we get:
   (W_{B})_{T}=(W_{M})_{T} or:
   Total (VO₂) consumed is proportional to [(M)×(MCDA)_{T-area}]
   In conventional (VO₂) measurements, oxygen consumption is expressed as VO₂/M. Thus by dividing each side of the proportionality by M, our final relationship is:
      Total (VO₂/M) is proportional to (MCDA)_{T-area}
17. A graph of Total (VO₂/M) versus (MCDA)_{T-area} for all the patients should be linear and follow the general equation Y−aX+b.
   VO₂ Max:
18. From (11) above based on a triangle's Area=½ BH, where:
   Area=total O₂ consumed
   B=time to VO₂ Max
   H 32 VO₂ Max, then:
   (Total O₂)=½ (Time to VO₂ Max) (VO₂ Max), or
   (VO₂Max)=[2(Total O₂)/(Time to VO₂ Max)]
19. A graph of (VO₂Max) versus [2(Total O₂)/(Time to VO₂Max)] for all the patients should be linear and follow the general equation Y=aX+b.
   Total Work:

Data of 8 treadmill patients indicated by a straight-line fit showed a correlation coefficient of 0.83. Additional studies may reduce the scatter and verify linearity.

VO₂Max:

Data of 7 treadmill patients indicated by a straight-line fit showed a correlation coefficient of 0.98. One patient was eliminated from data collection due to being unable to remain on the treadmill for sufficient time to reach VO₂Max. Treadmill-measured calorie expenditures contrasted with results derived from operation of the present inventions, follow:

Definitions: (Dimensional Analysis Included)

20. TEEM=Total Energy Expenditure Measurement (according to the present invention)
21. Acceleration (A)=Distance/Time²=(D)/(T)² with units in (cm/sec²)]
22. Force (F)=Mass×Acceleration=(M)(A) with units in [(g)(G)] or [(g)(CM/sec-²)]
23. Work (W)=Energy (E)=Force×Distance=(F)(D) (with units of ergs, calories) by substituting (22) into this equationfor (F) we obtain:
   23.1 E=(M)(A)(D) with units in [(g)(G)(cm)] or [(g)(cm²/sec²)]
24. Distance (D) on a treadmill is equal to time (T) of the test multiplied by the treadmill rate (R) thus D=(T)(R) or by substituting for (D) in equation 23.1 we get:
   24.1 E=(M)(A)(T)(R) with units in [(g)(G)(cm)] or [(g)(cm²/sec²)]
25. Maximum Change in Dynamic Acceleration (MCDA) is a mathematical treatment of the TEEM measured acceleration data, which measures acceleration values in G's, and is proportional to (A) thus:
   25.1 (A)=(a)(MCDA), where: (a) is a proportional constant. Then by substitution for (A) in equation 24.1 we get:
   25.2 E−(M)(a)(MCDA)(T)(R)
26. VO₂ is the measured oxygen consumption of an individual during an aerobic stress test and is expressed in ml/min or L/min.
   Conversion Factors and Test Conditions:
27. To convert from G's to cm/sec multiply by 981 (Ref 2 below)
28. To convert from ergs to kilocalories multiply by 2.39×10⁻¹¹ (Ref 2)
29. To convert from Liters of O₂ to kilocalories of energy multiply by 4.8 (Ref 1)
30. Treadmill rate of speed (R) was 13.4 cm/sec
31. Treadmill slope grade was 0.05
32. At rest energy expenditure, E_{R}=1 kcal/kg/hour or ER=1.67×10⁻² kcal/kg/min (Ref 1)
33. Total oxygen consumption, Total (VO₂), was obtained by summing the amount of oxygen consumed for each minute interval during the test. The amount of O₂ consumed for the last interval, which was usually less than a minute, was calculated by multiplying the factional portion of a minute times the last interval consumption rate.
   Energy Expenditure Calculation:
34. Total Maximum Change in Dynamic Acceleration α([(MCDA)ₐᵣₑₐ] is the sum of the area under each (α)(MCDA)(T) curve and is equal to the integral, ∫yᵢ dx, where yᵢ=height of a rectangle segment, (i), with infinitesimal base width, dx. After integration, [(MCDA)_{T-Area}] is equal to (Σyi)(x); or since: (Σyᵢ) is equal to (α)(MCDA) and (x) is equal to (T), then:
   34.1 (α)(MCDA)(T)=(α)[(MCDA)ₐᵣₑₐ]. Where: (α) (MCDA) is measured in G's and time (T) is measured in minutes. Then by substituting 34.1 into 25.2 we get the final equation:
      34.2E=(M)(α)[(MCDA)ₐᵣₑₐ](R).
      Converting from G's, ergs, kg and minutes we get energy in Kilocalories:
      34.3E (in kcal)=(981)(2.39×10⁻¹¹)(60)(10³)(α)(M)[(MCDA)ₐᵣₑₐ](R)
      Dimensional analysis of equation 34.3:
      E(in kcal)=(cm/sec²/G)(kcal/erg)(sec/min)(kg)(g/kg)(G)(min)(cm/sec).
      After unit cancellation (see 23.1 above): E=(g cm²/sec²)(kcal/erg)=kcal: Simplifying 34.3 when (R)=13.4 (cm/sec)(from 30 above) gives:
      34.4E(in kcal)=[1.89×10⁻²(α)(M)(MCDA)ₐᵣₑₐ]
      E is in kcal, (M) is in kg, (α) is unit less, (MCDA)ₐᵣₑₐ is in G's-min
35. Determination of energy expenditure on a treadmill from the TEEM according to the present invention:
Total energy expenditure (E_{T}) on a treadmill for a person of mass (M) is the sum of the rest component (R) plus the horizontal component (H) plus the vertical component (V):
35.1E_{T}=ΣE_{R}+E_{H}+E_{V}
For E_{R}:
35.2 From 32 above, E_{R}=(1.67×10⁻² kcal/kg/min)(M) where: (E_{R}) in kcal, (T) in minutes, (M) in kg
For E_{H} & E_{V}:
   Energy expenditure for (E_{H}) and (E_{V}) is recorded by the TEEM device and can be calculated from (34.4) above taking into account that (E_{V}) requires 18 times more calorie expenditure than (E_{H}) (ref 1).
35.3E_{H}=(1.89×10⁻²)(α)(M)(MCDA)ₐᵣₑₐ
The vertical portion of the treadmill is proportional to the percent grade and can be calculated from:

$$35.4 \begin{matrix}{E}_{V} = ( 18 ) ( \% grade ) {E}_{H} \\ = ( 18 ) ( \% grade ) [ ( 1.89 \times {10}^{- 2} ) ( α ) ( M ) {( MCDA )}_{area} ] \\ = ( 18 ) ( 0.05 ) ( 1.89 \times {10}^{- 2} ) ( α ) ( M ) {( MCDA )}_{area} \\ = ( 1.7 \times {10}^{- 2} ) ( α ) ( M ) {( MCDA )}_{area}\end{matrix}$$
Equations 35.3 and 35.4 can be combined and simplified to give:
   35.5E_{H}+E_{V}=E_{H+V}=(3.59×10⁻²)(α)(M)(MCDA)ₐᵣₑₐ
Then the final equation for energy expenditure measurement from the TEEM device:

$$35.6 \begin{matrix}{E}_{T} = Σ {E}_{R} + {E}_{H} + {E}_{V} \\ = Σ {E}_{R} + {E}_{H + V} \\ = ( 1.67 \times {10}^{- 2} ) ( T ) ( M ) + ( 3.59 \times {10}^{- 2} ) ( α ) ( M ) {( MCDA )}_{area}\end{matrix}$$
36. Determination of energy expenditure on a treadmill from oxygen consumption, VO2:
   36.1 E_{T} (in Kcal)=[(ΣVO₂)(4.8 Kcal/L)] where ΣVO₂ is total VO₂ in liters and 4.8 kcal/L is the conversion factor (obtained from ref 1 below).
37. Energy calculated from the TEEM device should equal the energy determined by oxygen consumption. Thus equating the two equations we get the equation:
   37.1E_{T}(VO₂)=E_{T(TEEM)}=ΣE_{R}+E_{H+V}
   Thus from 37.1 and 35.6 above:
      37.2(ΣVO₂)(4.8 Kcal/L)=(1.67×10⁻²)(T)(M)+(3.59×10⁻² )(α)(M)(MCDA)ₐᵣₑₐ
      Conclusion:
38. Graphing (ΣVO2) vs. (M)(MCDA)area or a rearrangement of terms will give a straight line. A simpler treatment assumes that since total consumed VO2 is directly proportional to energy in a biological system, then (MCDA)ₐᵣₑₐ is too since it records all body movement (including breathing). Then energy obtained from VO₂ can is equated to energy obtained from (MCDA)ₐᵣₑₐ to give:
   38.1[(ΣVO₂)×(4.8 Kcal/L)]=(MCDA)ₐᵣₑₐ
   Then graphing [(ΣVO₂)(4.8 Kcal/L)] Vs (MCDA)ₐᵣₑₐ or a rearrangement of terms will give a straight line.
      References:
1. Essentials of Cardiopulmonary Exercise Testing, Jonathan Meyers, PhD
2. Handbook of Chemistry and Physics, CRC

1. Apparatus for detecting an individual's physical condition during an activity, the apparatus comprising:
a processor connected to receive data indicative of selected parameters of the individual's condition, including at least, heart rate;
a plurality of accelerometers disposed to align substantially along orthogonal axes of the individual's movements during the activity for supplying data indication of such accelerations to the processor' and
an output device connected to the processor for providing sensory output indication of calculations by the processor from the data supplied thereto that is indicative of parameters representative of the individual's physical condition.

2. A method for analyzing parameters indicative of a physical condition of an individual, comprising the steps for:
sensing physical data including heart rate;
sensing acceleration data of the individual along a plurality of acceleration axes;
filtering the acceleration data to yield a selected component thereof; and
integrating the selected component of acceleration data to. produce an output indicative of an individual's physical condition.
